(19) United States
(12) Patent Application Publication
(10) Pub. No.: US 2024/0392523 A1
(43) Pub. Date: Nov. 28, 2024

(54) System for Monitoring Sliding Soil Mass

(71) Applicant: Pillar Innovations, LLC, Grantsville, MD (US)

(72) Inventors: Avery Simon, Grantsville, MD (US); Brett Dugan, Grantsville, MD (US)

(21) Appl. No.: 18/672,848

(22) Filed: May 23, 2024

Related U.S. Application Data
(60) Provisional application No. 63/468,455, filed on May 23, 2023.

(51) Int. Cl. E02D 1/02

(52) U.S. Cl. CPC E02D 1/022

(57) ABSTRACT

A system for monitoring sliding soil mass includes a plurality of sensor assemblies, with each sensor assembly including at least one sensor subassembly and a node subassembly in electronic communication with the at least one sensor subassembly, with the node subassembly including a power source, data storage, and a communication device, and with each sensor subassembly including a sensor configured to detect soil movement when the sensor subassembly is positioned within a soil mass, and a supernode in electronic communication with at least one of the plurality of sensor assemblies. The supernode is configured to transmit data from the plurality of sensor assemblies to a local and/or remote device.

### CROSS-REFERENCE TO RELATED APPLICATION

The present application claims the benefit of U.S. Provisional Application Ser. No. 63/468,455, filed May 23, 2023, which is hereby incorporated by reference in its entirety.

### BACKGROUND OF THE INVENTION

### Field of the Invention

The present application relates to a system for monitoring sliding soil mass.

### Description of Related Art

One of the largest threats to safe gathering, transmission and distribution of pipeline product is rupture from a pipeline's engagement in a geohazard. Operators currently monitoring the movement and potential pipeline engagement utilize in situ instrumentation, such as shape arrays or inclinometers. The installation of shape arrays requires operators to obtain expensive permitting and mobilize heavy and expensive machinery to drill and install the borehole casing required to provide near real time monitoring of geohazards.

### SUMMARY OF THE INVENTION

In one aspect or embodiment, a system for monitoring sliding soil mass includes a plurality of sensor assemblies, with each sensor assembly including at least one sensor subassembly and a node subassembly in electronic communication with the at least one sensor subassembly, with the node subassembly including a power source, data storage, and a communication device, and with each sensor subassembly including a sensor configured to detect soil movement when the sensor subassembly is positioned within a soil mass, and a supernode in electronic communication with at least one of the plurality of sensor assemblies. The supernode is configured to transmit data from the plurality of sensor assemblies to a local and/or remote device.

The sensor subassembly may include at least one of a temperature sensor, a strain gauge, and an accelerometer. The node subassembly may include a photovoltaic cell and a battery. The node subassembly may include a microcontroller. Each sensor subassembly may be configured to be positioned within a borehole formed in a soil mass, where the node subassembly is configured to be positioned above the borehole. The plurality of sensor assemblies may each include a plurality of sensor subassemblies, with the sensor subassemblies connected in series for each sensor assembly.

Each of the sensor subassemblies may include a first keyed connector positioned at a first end of the sensor subassembly and a second keyed connector positioned at a second end of the sensor subassembly. The first keyed connector may be a male connector and the second keyed connector may be a female connector. Each of the sensor subassemblies may include an overmold positioned over at least a portion of the first keyed connector and the second keyed connector. Each of the sensor subassemblies may include a strain relief.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a sensor assembly according to one embodiment of the present application;

FIG. 2 is a perspective view of a sensor assembly according to one embodiment of the present application;

FIG. 3 is a top view of a sensor assembly according to one embodiment of the present application;

FIG. 4 is a schematic view of a node subassembly according to one embodiment of the present application;

FIG. 5 is a schematic view of a sensor subassembly according to one embodiment of the present application;

FIG. 6 is a schematic view of a sensor subassembly according to one embodiment of the present application;

FIG. 7 is a schematic view of a system of monitoring sliding soil mass;

FIG. 8 is a perspective view of a sensor subassembly according to one embodiment of the present application; and

FIG. 9 is a partial perspective view a sensor subassembly according to one embodiment of the present application.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

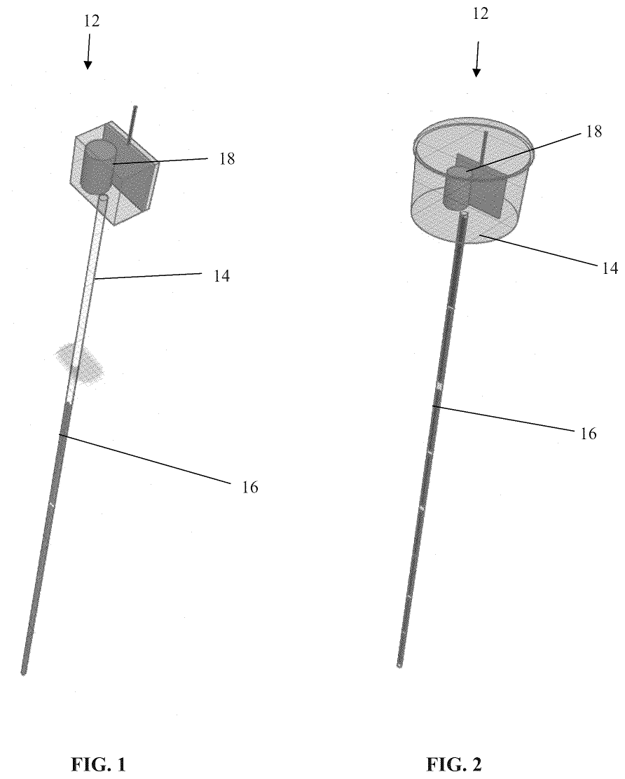

Referring to FIGS. 1-7, a system 10 for monitoring sliding soil mass includes a plurality of sensor assemblies 12, with each sensor assembly 12 including a housing 14, at least one sensor subassembly 16, and a node subassembly 18 in electronic communication with the at least one sensor subassembly 16, with the node subassembly 18 including a power source, data storage, and a communication device. The system 10 further includes a supernode 20 in electronic communication with the plurality of sensor assemblies 12, with the supernode 20 configured to transmit data from the plurality of sensor assemblies 12 to a local and/or remote device. In one aspect or embodiment, one or more sensor assemblies 12 are provided. In some aspects or embodiments, three sensor assemblies 12 are provided at spaced apart locations. The sensor assemblies 12 are configured be driven into the soil via light hand tools. The senor subassembly 16 may include accelerometers and/or magnetometers in spaced apart locations along a length of the housing.

The system 10 is configured to enable operators to have agile and inexpensive monitoring capabilities for depth and magnitude of soil movement so that geohazard engagement can be inferred in near real time. The node subassembly 18 handles communication to the sensor assemblies 12 and telemetry device/data storage in addition to power storage. The sensor subassemblies 16 are connected in series to provide a customizable length configuration, with each sensor subassembly 16 being configured according to its depth for depth and magnitude calculations. In some aspects or embodiments, the sensor assemblies 12 form a mesh network for communication with the supernode 20. The supernode 20 is configured to be placed at a higher elevation relative to the sensor assemblies 12 such that sensor assemblies 12 at lower elevations and out of telemetry with the supernode 20 are configured to communicate with other sensor assemblies 12 and from those other sensor assemblies 12 to the supernode 20 thereby providing real time communication from the sensor assemblies 12 to a network, the cloud, or other communication arrangement.

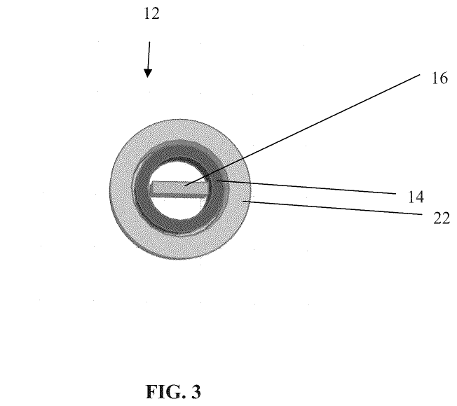

Referring to FIGS. 1-3, each sensor assembly 12 may be an above ground installation (FIG. 1) with a portion of the sensor assembly 12 positioned above a ground surface or a flush mount installation with a top of each sensor assembly 12 flush with a ground surface. As shown in FIG. 3, the sensor assembly 12 may be installed using dynamic cone penetration 22 that is subsequently removed leaving the sensor assembly 12 in place. The dynamic cone penetration 22 may be hollow, with the dynamic cone penetration 22 inserted into the ground to a desired depth and the sensor assembly 16 insertion within the hollow tube of the dynamic cone penetration 22. The dynamic cone penetration 22 can be removed while leaving the sensor assembly 12 in place. In some aspects or embodiments, the sensor assembly 12 includes one or more anchors, such as a toggle anchor, that engage the soil structure upon upward movement of the sensor assembly 12. While FIGS. 1 and 2 show the sensor subassemblies 16 received within the housing 14, each of the sensor subassemblies 16 may include their own housing and may be connected in series and inserted within the dynamic cone penetration 22 without a separate housing 14 that receives all of the connected sensor subassemblies 16.

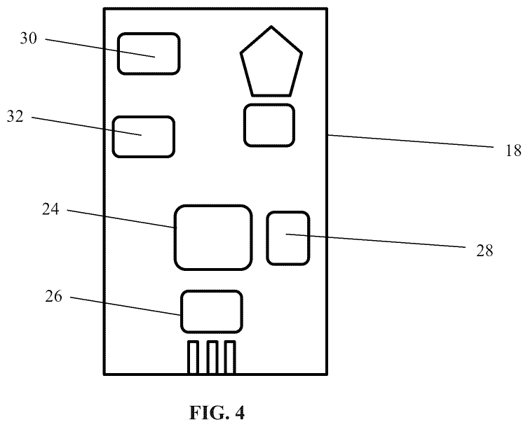

Referring to FIG. 4, in one aspect or embodiment, each node subassembly 18 includes a microcontroller 24 based circuit to collect and process site sensor readings. The node subassembly 18 includes a one wire sensor interface 26 that can address and interface with multiple types and quantities of sensors. The node subassembly 18 utilizes a 900 Mhz node-to-node and node-to-base communication 28 with a low power design configured to utilize various power supply options, including, but not limited to, photovoltaic 30 and battery power 32. The node subassembly 18 is configured to have a small footprint.

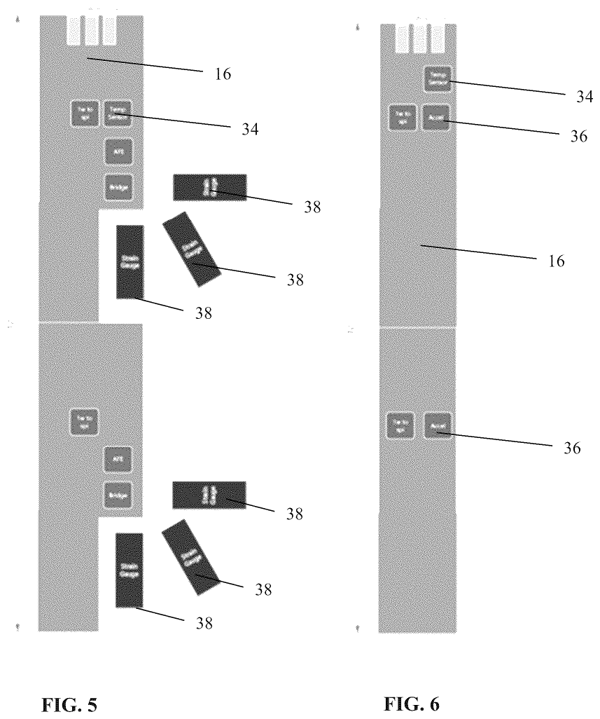

Referring to FIGS. 5 and 6, in one aspect or embodiment, the sensor subassemblies 16 may each include a flexible circuit board(s) to function under deformation and may be modular in design to allow configurable lengths. In some aspects or embodiments, the sensor subassemblies 16 include a temperature sensor 34 for thermal compensation. Each sensor subassembly 16 is configured to sense gravitational acceleration (tilt) and/or strain via accelerometer(s) 36 and strain gauge(s) 38 or other suitable sensors. The sensor subassembly 16 may only sense strain (FIG. 5) or may only sense tilt (FIG. 6).

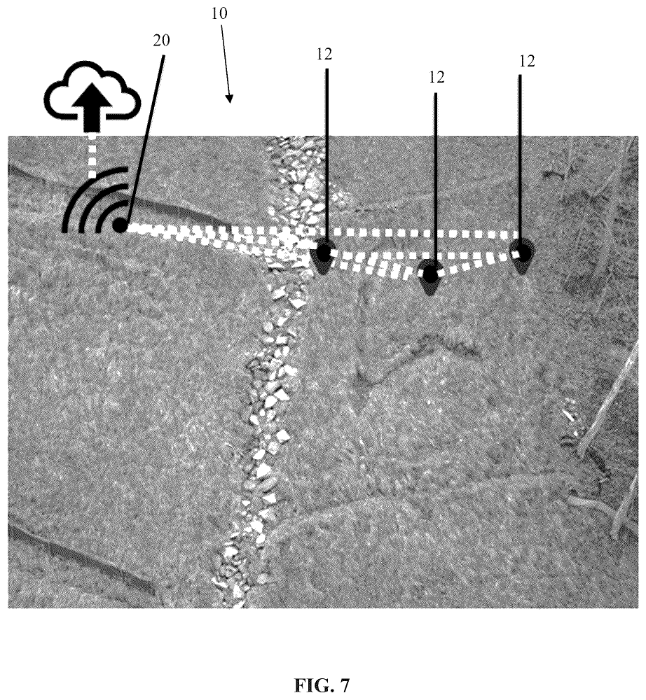

Referring to FIG. 7, in one aspect or embodiment, the system 10 includes two or more sensor assemblies 12 (shown with three sensor assemblies) that are in close proximity to each other, such as within 50, 25, 10, 5, or two feet within each other. The sensor assemblies 12 communicate locally to the supernode 20, which communicates to a remote and/or local device, such as a mobile device, computer, or other suitable device or system. The supernode 20 may communicate with a remote device via a cellular or other wireless connection or network.

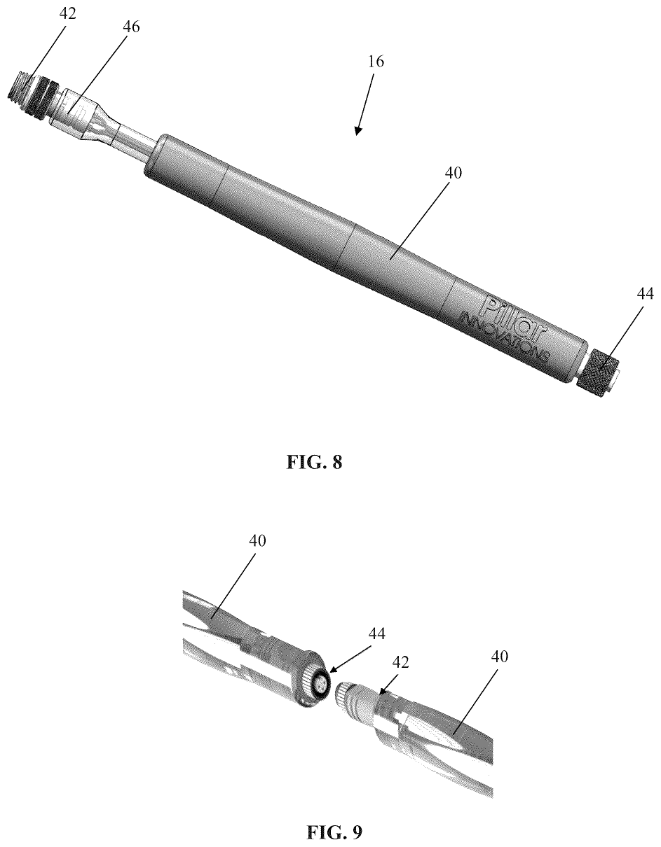

Referring to FIGS. 8 and 9, in some aspects or embodiments, the sensor subassembly 16 includes an overmold 40 configured to provide protection of water instruction and/or provide additional mechanical strength to be able to withstand forces from the soil. Although not shown, the overmold 40 may include a first mold applied at low pressure over the printed circuit board, with a second mold applied at high pressure over the first mold. The sensor subassembly 16 may also include corresponding keyed connectors 42, 44 at each end of the sensor subassembly 16 encapsulated by the overmold 40. The keyed connectors 42, 44 may be male and female connectors having an alignment key and corresponding protrusion to ensure alignment of the sensor subassembly 16 after connection of one sensor subassembly 16 to another sensor subassembly 16. The keyed connectors 42, 44 may include corresponding threaded portions to ensure a secure connection between the respective connectors 42, 44. The keyed connectors 42, 44 and their alignment with the other components of the sensor subassembly 16 are configured to maintain alignment for direction resolution of landslide movement sensed by the sensor subassembly 16. In some aspects or embodiments, the sensor subassembly 16 may include a strain relief 46 configured to provide flexibility between the sensor subassemblies 16 to ensure the sensor subassemblies 16 engage with a landslide or soil movement and provide an accurate indication of a landslide movement.

The system 10 is configured to measure alignment of installation through geomagnetic field and measure tilt of sections. The system 10 is configured to be installed with light tools that fit within a backpack. The system 10 does not require expensive casing to be installed prior to installation of the system 10. The system 10 is configured to provide local reading storage or telemetry for near real-time readings. The system 10 is configured to utilize sensor agnostic board design after communication bus and a flexible circuit board.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A system for monitoring sliding soil mass comprising:
a plurality of sensor assemblies, each sensor assembly comprising at least one sensor subassembly and a node subassembly in electronic communication with the at least one sensor subassembly, the node subassembly comprising a power source, data storage, and a communication device, each sensor subassembly comprising a sensor configured to detect soil movement when the sensor subassembly is positioned within a soil mass; and
a supernode in electronic communication with at least one of the plurality of sensor assemblies, the supernode configured to transmit data from the plurality of sensor assemblies to a local and/or remote device.

2. The system of claim 1, wherein the sensor subassembly comprises at least one of a temperature sensor, a strain gauge, and an accelerometer.

3. The system of claim 1, wherein the node subassembly comprises a photovoltaic cell and a battery.

4. The system of claim 1, wherein the node subassembly comprises a microcontroller.

5. The system of claim 1, wherein each sensor subassembly is configured to be positioned within a borehole formed in a soil mass, and wherein the node subassembly is configured to be positioned above the borehole.

6. The system of claim 1, wherein the plurality of sensor assemblies each comprise a plurality of sensor subassemblies, with the sensor subassemblies connected in series for each sensor assembly.

7. The system of claim 1, wherein each of the sensor subassemblies comprises a first keyed connector positioned at a first end of the sensor subassembly and a second keyed connector positioned at a second end of the sensor subassembly.

8. The system of claim 7, wherein the first keyed connector is a male connector and the second keyed connector is a female connector.

9. The system of claim 8, wherein each of the sensor subassemblies comprises an overmold positioned over at least a portion of the first keyed connector and the second keyed connector.

10. The system of claim 9, where each of the sensor subassemblies comprises a strain relief.
